(19) United States
(12) Patent Application Publication
(10) Pub. No.: US 2018/0030508 A1
(43) Pub. Date: Feb. 1, 2018

(54) NUCLEIC ACID AMPLIFICATION REACTION METHOD, REAGENT SET, AND METHOD OF USING REAGENT SET

(71) Applicant: Seiko Epson Corporation, Tokyo (JP)

(72) Inventors: Kotaro IDEGAMI, Chino (JP); Masayuki UEHARA, Matsumoto (JP); Masato HANAMURA, Shiojiri (JP)

(21) Appl. No.: 15/650,015

(22) Filed: Jul. 14, 2017

(30) Foreign Application Priority Data
Jul. 29, 2016 (JP) 2016-149492

(51) Int. Cl. C12Q 1/68; G01N 27/447

(52) U.S. Cl. CPC C12Q 1/6806; C12Q 1/686; G01N 27/447; G01N 2001/4038

(57) ABSTRACT

A nucleic acid amplification reaction method includes a thermal cycling step of performing thermal cycling for amplifying a nucleic acid for a reaction solution containing a primer, a temperature increasing step of increasing the temperature of the reaction solution to a temperature at which the amplified nucleic acid is denatured after the thermal cycling step, a temperature decreasing step of decreasing the temperature of the reaction solution to a temperature at which a probe hybridizes after the temperature increasing step, and a probe addition step of adding the probe to the reaction solution.

### BACKGROUND

### 1. Technical Field

The present invention relates to a nucleic acid amplification reaction method, a reagent set, and a method of using a reagent set.

### 2. Related Art

In recent years, due to the development of technologies utilizing genes, medical treatments utilizing genes such as gene diagnosis or gene therapy have been drawing attention. In addition, many methods using genes in determination of breed varieties or breed improvement have also been developed in agriculture and livestock industries. As technologies for utilizing genes, technologies such as a PCR (Polymerase Chain Reaction) method are widely used. Nowadays, the PCR method has become an indispensable technology for elucidation of information on biological materials.

The PCR method is a method of amplifying a target nucleic acid by performing thermal cycling for a solution (reaction solution) containing a nucleic acid to be amplified (target nucleic acid) and a reagent. The thermal cycling is a treatment of periodically subjecting the reaction solution to two or more temperature steps. In the PCR method, a method of performing two- or three-step thermal cycling is generally used. In the amplification of a nucleic acid, for example, quantitative determination can be performed using a probe.

An increase in PCR speed is a necessary technology for reducing the testing time of a genetic test, and has been much expected in the genetic testing industries.

For example, JP-T-2015-520614 (Patent Document 1) discloses a method in which a polymerase is provided at a concentration of at least 0.5 μM and a primer is provided at a concentration of at least 2 μM, and a cycle is completed in a cycle time of less than 20 seconds per cycle.

As a result of intensive studies, the present inventors found that when the thermal cycling speed is increased (the thermal cycling time is reduced) for increasing the PCR speed, a nucleic acid amplification reaction is inhibited by a probe.

### SUMMARY

An advantage of some aspects of the invention is to provide a nucleic acid amplification reaction method capable of increasing the thermal cycling speed. Another advantage of some aspects of the invention is to provide a reagent set capable of increasing the thermal cycling speed, and a method of using the same.

A nucleic acid amplification reaction method according to an aspect of the invention includes a thermal cycling step of performing thermal cycling for amplifying a nucleic acid for a reaction solution containing a primer, a temperature increasing step of increasing the temperature of the reaction solution to a temperature at which the amplified nucleic acid is denatured after the thermal cycling step, a temperature decreasing step of decreasing the temperature of the reaction solution to a temperature at which a probe hybridizes after the temperature increasing step, and a probe addition step of adding the probe to the reaction solution, wherein in the thermal cycling step, the time per cycle of the thermal cycling is 9 seconds or less, the Tm value of the primer is 70° C. or higher and 80° C. or lower, the probe addition step is performed after the thermal cycling step and before the temperature increasing step, or after the temperature increasing step and before the temperature decreasing step, or after the temperature decreasing step.

According to such a nucleic acid amplification reaction method, after a nucleic acid is amplified in a reaction solution, a probe is added to the reaction solution, and therefore, inhibition of amplification of the nucleic acid by the probe can be suppressed. Therefore, according to such a nucleic acid amplification reaction method, the thermal cycling speed can be increased (see the below-mentioned “3. Experimental Examples” for the details).

In the nucleic acid amplification reaction method according to the aspect of the invention, the time per cycle of the thermal cycling may be 2.5 seconds or less.

According to such a nucleic acid amplification reaction method, the thermal cycling speed can be increased (see the below-mentioned “3. Experimental Examples” for the details).

In the nucleic acid amplification reaction method according to the aspect of the invention, the probe may contain a minor groove binder molecule.

According to such a nucleic acid amplification reaction method, the fluorescence intensity from the probe can be increased, and therefore, the amplification amount can be quantitatively determined with high sensitivity (see the below-mentioned “3. Experimental Examples” for the details).

In the nucleic acid amplification reaction method according to the aspect of the invention, the reaction solution may contain a divalent cation, and the concentration of the divalent cation contained in the reaction solution may be 2 mM or more and 7.5 mM or less.

According to such a nucleic acid amplification reaction method, while accelerating an elongation reaction by a polymerase and increasing the PCR speed, nonspecific amplification is suppressed, and a decrease in yield of a specific amplification product can be suppressed.

In the nucleic acid amplification reaction method according to the aspect of the invention, the reaction solution may contain MgCl₂, the divalent cation may be derived from MgCl₂, and the concentration of MgCl₂ contained in the reaction solution after the probe is added may be 4 mM or more and 7.5 mM or less.

According to such a nucleic acid amplification reaction method, while increasing the thermal cycling speed, nonspecific amplification is suppressed, and a decrease in yield of a specific amplification product can be suppressed.

In the nucleic acid amplification reaction method according to the aspect of the invention, the reaction solution may contain MgSO₄, the divalent cation may be derived from MgSO₄, and the concentration of MgSO₄ contained in the reaction solution after the probe is added may be 2 mM or more and 3 mM or less.

According to such a nucleic acid amplification reaction method, while increasing the thermal cycling speed, nonspecific amplification is suppressed, and a decrease in yield of a specific amplification product can be suppressed.

In such a nucleic acid amplification reaction method, an optimal concentration range of the divalent cation for suppressing nonspecific amplification and suppressing the decrease in yield of a specific amplification product while accelerating an elongation reaction and increasing the PCR speed varies depending on the type of the divalent cation.

In the nucleic acid amplification reaction method according to the aspect of the invention, the probe may be a non-hydrolysis probe.

According to such a nucleic acid amplification reaction method, it is not necessary to degrade the probe in the elongation reaction, and therefore, there is no need to provide an amplification region to which the probe anneals between the forward primer and the reverse primer. According to this, it becomes possible to design the amplification region narrower than in a hydrolysis-type system. Since the amplification region becomes narrower, the annealing time can be reduced, and thus, the thermal cycling speed can be increased.

In the nucleic acid amplification reaction method according to the aspect of the invention, with respect to the amount of the primer which anneals to a strand to which the probe hybridizes in the nucleic acid, the ratio of the amount of the primer which anneals to a strand complementary to the strand in the nucleic acid may be more than 1 and less than 4.

According to such a nucleic acid amplification reaction method, the fluorescence intensity from the probe can be increased, and therefore, the amplification amount can be quantitatively determined with high sensitivity (see the below-mentioned “3. Experimental Examples” for the details).

In the nucleic acid amplification reaction method according to the aspect of the invention, in the temperature decreasing step, the speed of decreasing the temperature of the reaction solution may be 2.5° C./s or more.

According to such a nucleic acid amplification reaction method, the fluorescence intensity from the probe can be increased, and therefore, the amplification amount can be quantitatively determined with high sensitivity (see the below-mentioned “3. Experimental Examples” for the details).

A reagent set according to an aspect of the invention includes a first reagent which is a reagent for amplifying a nucleic acid and contains a primer and MgCl₂, and a second reagent which is a reagent for quantitatively determining the amplification amount of a nucleic acid and contains a probe, wherein the first reagent and the second reagent are separated from each other, the Tm value of the primer is 70° C. or higher and 80° C. or lower, and the first reagent becomes a reaction solution for performing a nucleic acid amplification reaction, and when the second reagent is added to the reaction solution, the concentration of MgCl₂ contained in the reaction solution is 4 mM or more and 7.5 mM or less.

According to such a reagent set, the thermal cycling speed can be increased.

A reagent set according to an aspect of the invention includes a first reagent which is a reagent for amplifying a nucleic acid and contains a primer and MgSO₄, and a second reagent which is a reagent for quantitatively determining the amplification amount of a nucleic acid and contains a probe, wherein the first reagent and the second reagent are separated from each other, the Tm value of the primer is 70° C. or higher and 80° C. or lower, and the first reagent becomes a reaction solution for performing a nucleic acid amplification reaction, and when the second reagent is added to the reaction solution, the concentration of MgSO₄ contained in the reaction solution is 2 mM or more and 3 mM or less.

According to such a reagent set, the thermal cycling speed can be increased.

A method of using a reagent set according to an aspect of the invention is a method of using the reagent set according to the aspect of the invention, including preparing the reaction solution by bringing the first reagent and a template nucleic acid solution containing a template nucleic acid into contact with each other, and after a nucleic acid is amplified in the reaction solution, adding the second reagent to the reaction solution.

According to such a method of using a reagent set, the thermal cycling speed can be increased.

### BRIEF DESCRIPTION OF THE DRAWINGS

The invention will be described with reference to the accompanying drawings, wherein like numbers reference like elements.

FIG. 1 is a flowchart for illustrating a nucleic acid amplification reaction method according to an embodiment.

FIG. 2 is a cross-sectional view schematically showing a thermal cycler for performing thermal cycling for a reaction solution according to an embodiment.

FIG. 3 is a graph showing a fluorescence intensity.

FIG. 4 is a graph showing a fluorescence intensity.

FIG. 5 shows the results of electrophoresis.

FIG. 6 is a graph showing a fluorescence intensity.

FIG. 7 is a graph showing a fluorescence intensity.

FIG. 8 is a graph showing a fluorescence intensity.

FIG. 9 is a graph showing a fluorescence intensity.

FIG. 10 is a graph showing a fluorescence intensity.

FIG. 11 is a graph showing a relationship between the reaction time of PCR and a fluorescent brightness.

FIG. 12 is a graph showing a relationship between the reaction time of PCR and a fluorescent brightness.

### DESCRIPTION OF EXEMPLARY EMBODIMENTS

Hereinafter, preferred embodiments of the invention will be described in detail with reference to the accompanying drawings. Note that the embodiments described below are not intended to unduly limit the content of the invention described in the appended claims. Further, all the configurations described below are not necessarily essential components according to the invention.

### 1. Reagent Set

First, a reagent set according to this embodiment will be described. The reagent set according to this embodiment includes a first reagent and a second reagent. The first reagent and the second reagent are separated from each other. The first reagent and the second reagent may be in a liquid form or may be in a lyophilized state. The first reagent and the second reagent may be housed in one case body in a state of being separated from each other.

### 1.1. First Reagent

The first reagent is a reagent for amplifying a nucleic acid. The first reagent contains primers, a polymerase, dNTP, and a buffer.

### 1.1.1. Primers

The primer is designed to anneal to a template nucleic acid (template). The “anneal (annealing)” refers to an action (a phenomenon) in which a primer binds to a DNA. The first reagent contains a forward primer which anneals to one template nucleic acid having a single-stranded structure (single-stranded DNA) after a template nucleic acid having a double-stranded structure (double-stranded DNA) is denatured, and a reverse primer which anneals to the other single-stranded DNA as the primers. The concentrations of the forward primer and the reverse primer contained in the reaction solution after the second reagent is added are each, for example, 0.4 μM or more and 12.8 μM or less, preferably 1.6 μM or more and 9.6 μM or less.

The “reaction solution” refers to a solution for performing a nucleic acid amplification reaction formed by bringing a template nucleic acid solution containing a DNA (deoxyribonucleic acid) or an RNA (ribonucleic acid) and the first reagent into contact with each other.

With respect to the amount of the primer which anneals to a strand to which a probe hybridizes in the nucleic acid, the ratio of the amount of the primer which anneals to a strand complementary to the strand in the nucleic acid may be more than 1 and less than 4. For example, in the case where a probe hybridizes to a first single-stranded DNA, the forward primer anneals to the first single-stranded DNA, and the reverse primer anneals to a second single-stranded DNA complementary to the first single-stranded DNA, the ratio of the amount of the reverse primer to the amount of the forward primer may be more than 1 and less than 4. By setting the ratio within the above range, when the second reagent containing the probe is added to a reaction container, the emission intensity (fluorescence intensity) from the probe can be increased, and therefore, the amplification amount can be quantitatively determined with high sensitivity (see the below-mentioned “3. Experimental Examples” for the details).

The Tm value of the primer (the forward primer and the reverse primer) is 70° C. or higher and 80° C. or lower, preferably 70° C. or higher and 75° C. or lower. According to this, the reagent set according to this embodiment can increase the thermal cycling speed (see the below-mentioned “3. Experimental Examples” for the details). The Tm value is an index of the temperature at which a primer anneals to a template nucleic acid, and is a temperature at which 50% of a double-stranded DNA is dissociated into single-stranded DNAs, that is, a melting temperature. If the temperature is not lower than the Tm value, not less than half of the primer anneals to the template nucleic acid. The Tm value of the forward primer and the Tm value of the reverse primer may be the same as or different from each other.

As a calculation method of the Tm value, for example, a nearest neighbor method is exemplified, and the Tm value can be calculated according to the following formula (1). In the invention, in the case where calculation is performed according to the following formula (1), the Tm value of the primer falls within a range of 70° C. or higher and 80° C. or lower.

Tm=1000 ΔH/(−10.8 +ΔS+R×ln(Ct/4))−273.15+16.6 log[Na⁺] (1)

In the formula (1), ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primers, and Na⁺ represents the concentration (mol/L) of a monovalent cation contained in the buffer.

The primer may contain an artificial nucleic acid. According to this, the Tm value of the primer can be made to fall within the above range without increasing the number of bases of the primer. When the number of bases of the primer is increased, nonspecific adsorption occurs or the primers form a primer dimer (double strand) , and a target nucleic acid cannot be amplified in some cases. As the primer dimer, there are a self-dimer which forms a double strand in one primer, and a cross-dimer which forms a double strand between the forward primer and the reverse primer.

The “artificial nucleic acid” refers to a nucleic acid molecule which can bind to a base of a DNA or an RNA through a hydrogen bond and is other than natural nucleic acid molecules.

Examples of the artificial nucleic acid include a 2′,4′-BNA (2′-O, 4′-C-methano-bridged nucleic acid, also known as “LNA (Locked Nucleic Acid)”) in which the oxygen atom at the 2′-position of a ribose ring of a nucleic acid is methylene-crosslinked to the carbon atom at the 4′-position, an ENA (2′-O, 4′-C-Ethylene-bridged Nucleic Acid), and a PNA (Peptide Nucleic Acid).

### 1.1.2. Polymerase

The polymerase is not particularly limited, however, examples thereof include a DNA polymerase. The DNA polymerase polymerizes nucleotides complementary to the bases of a template nucleic acid at the end of the primer annealing to the template nucleic acid having a single-stranded structure (single-stranded DNA). The DNA polymerase is preferably a heat-resistant enzyme or an enzyme for PCR, and there are a large number of commercially available products, for example, Taq polymerase, KOD polymerase, Tfipolymerase, Tth polymerase, modified forms thereof, and the like, however, a DNA polymerase capable of performing hot start is preferred. As the polymerase, there are a hydrolysis-type polymerase which degrades a probe by hydrolysis such as Taq polymerase, and a non-hydrolysis-type polymerase which does not degrade a probe by hydrolysis such as KOD polymerase. The KOD polymerase is derived from Thermococcus kodakarensis KOD1 and is a DNA polymerase from the genus Thermococcus. The amount of the polymerase contained in the reaction solution after the second reagent is added is, for example, 0.5 U or more.

1.1.3. dNTP

The dNTP refers to a mixture of four types of deoxyribonucleotide triphosphates. That is, the dNTP refers to a mixture of dATP, dCTP, dGTP, and dTTP. The DNA polymerase forms a new DNA by joining dATP, dCTP, dGTP, or dTTP to the end of the primer annealing to the template (an elongation reaction). The concentration of the dNTP contained in the reaction solution after the second reagent is added is, for example, 0.06 mM or more and 0.75 mM or less, preferably 0.125 mM or more and 0.5 mM or less.

### 1.1.4. Buffer

The buffer is, for example, a buffering agent containing a salt. Examples of the salt contained in the buffer include salts such as Tris, HEPES, PIPES, and phosphates. By using such a salt, the pH of the buffer can be adjusted.

The buffer contains a divalent cation. Examples of the divalent cation include Mn, Co, and Mg. In the case where the nucleic acid amplification reaction reagent becomes a reaction solution for performing a nucleic acid amplification reaction, the concentration of the divalent cation contained in the reaction solution is 2 mM or more and 7.5 mM or less.

By setting the concentration of the divalent cation to 2 mM or more, the elongation reaction by the polymerase is accelerated, and the PCR speed can be increased (specifically, the time per cycle of the thermal cycling can be reduced to 9 seconds or less). By setting the concentration of the divalent cation to 7.5 mM or less, nonspecific amplification is suppressed, and a decrease in yield of a specific amplification product can be suppressed.

Specifically, the buffer contains a divalent cationic compound, KCl, and Tris. More specifically, the buffer contains MgCl₂, and the divalent cation is derived from MgCl₂. That is, the divalent cation is produced by ionization of MgCl₂. In the case where the divalent cation is derived from MgCl₂, the concentration of Mg²⁺ is attributed to the activity of the polymerase. In the case where the nucleic acid amplification reaction reagent becomes a reaction solution for performing a nucleic acid amplification reaction, the concentration of MgCl₂ contained in the reaction solution is 4 mM or more and 7.5 mM or less, preferably 5 mM or more and mM or less, more preferably 5 mM. By setting the concentration of MgCl₂ to 4 mM or more, the elongation reaction by the polymerase is accelerated, and the PCR speed can be increased. By setting the concentration of MgCl₂ to 7.5 mM or less, nonspecific amplification is suppressed, and a decrease in yield of a specific amplification product can be suppressed. When the nucleic acid amplification reaction reagent is in a lyophilized state, the nucleic acid amplification reaction reagent is in a solid state, and contains MgCl₂, KCl, Tris, and an excipient such as trehalose.

The divalent cationic compound may be derived from MgSO₄. In this case, the buffer contains MgSO₄ in place of MgCl₂, and in the case where the nucleic acid amplification reaction reagent becomes a reaction solution for performing a nucleic acid amplification reaction, the concentration of MgSO₄ contained in the reaction solution is 2 mM or more and 3 mM or less, more preferably 2 mM. By setting the concentration of MgSO₄ to 2 mM or more, the elongation reaction by the polymerase is accelerated, and the PCR speed can be increased. By setting the concentration of MgSO₄ to 3 mM or less, nonspecific amplification is suppressed, and a decrease in yield of a specific amplification product can be suppressed.

### 1.1.5. Other Components

In the case where an RNA is used as the template nucleic acid, the first reagent further contains a reverse transcriptase. As the reverse transcriptase, for example, a reverse transcriptase derived from avian myeloblast virus, Ras-associated virus type 2, mouse Moloney murine leukemia virus, or human immunodefficiency virus type 1 is used.

In the case where the first reagent is lyophilized, the first reagent (lyophilized reagent) contains a sugar. Examples of the sugar include sucrose, trehalose, raffinose, and melezitose, each of which is a non-reducing sugar, among disaccharides and trisaccharides. Among the disaccharides and trisaccharides, particularly trehalose is preferably used because the function as a cryoprotective agent is high. Trehalose prevents the lyophilized reagent from coming into contact with a water molecule by its strong hydration force, and thus can improve the storage stability of the lyophilized reagent. The lyophilized reagent can be prepared by lyophilizing a mixed reagent solution containing the respective components of the nucleic acid amplification reaction reagent and a sugar. The temperature during lyophilization is, for example, about −80° C.

### 1.2. Second Reagent

The second reagent is a reagent for quantitatively determining the amplification amount of a nucleic acid. The second reagent contains a probe. The second reagent may contain a component other than the probe, or may be constituted by only the probe.

The probe is a fluorescently labeled probe to be used for quantitatively determining the amplification amount of a nucleic acid. When the second reagent is added to the reaction solution, the concentration of the probe contained in the reaction solution is 0.5 μM or more and 7.2 μM or less, preferably 0.9 μM or more and 3.6 μM or less.

The probe may be a hydrolysis probe containing a reporter dye and a quencher dye. Specifically, the probe may be TaqMan (registered trademark) probe. While the hydrolysis probe hybridizes to a single-stranded DNA to form a double-stranded structure, the light emission of a reporter dye is suppressed by a quencher dye (by a quenching effect) which is in close proximity to the reporter dye. However, when the probe is degraded by the exonuclease activity of the polymerase, the quenching effect is cancelled, and therefore, the reporter dye emits light. By this light emission, the amplification amount of a nucleic acid can be quantitatively determined. The “hybridization” refers to a phenomenon in which a probe binds to a DNA. In the case where a hydrolysis probe is used as the probe, Taq polymerase is used as the polymerase.

The probe may be a non-hydrolysis probe other than the hydrolysis probe. Specifically, the probe may be a Q (Quenching) probe utilizing a fluorescence-quenching phenomenon. The Q probe emits light in a state where it does not hybridize to a single-stranded DNA, and quenches the light when it hybridizes to a single-stranded DNA. By this difference in the emission intensity, the amplification amount of a nucleic acid can be quantitatively determined. In the case where the Q probe is used as the probe, KOD polymerase is used as the polymerase. The elongation reaction rate of KOD polymerase is larger than that of Taq polymerase, and therefore, KOD polymerase can increase the thermal cycling speed.

In the case where the probe is a non-hydrolysis probe, it is not necessary to degrade the probe in the elongation reaction, and therefore, there is no need to provide an amplification region to which the probe anneals between the forward primer and the reverse primer. According to this, it becomes possible to design the amplification region narrower than in a hydrolysis-type system. Since the amplification region becomes narrower, the annealing time can be reduced, and thus, the thermal cycling speed can be increased.

It is preferred that the probe contains a minor groove binder (MGB) molecule. According to this configuration, the reagent set according to this embodiment can increase the fluorescence intensity from the probe, and therefore, the amplification amount can be quantitatively determined with high sensitivity (see the below-mentioned “3. Experimental Examples” for the details).

The probe may contain an artificial nucleic acid. As the artificial nucleic acid, the above-mentioned artificial nucleic acid can be used.

### 1.3. Using Method

Next, the method of using the reagent set according to this embodiment will be described. In the method of using the reagent set according to this embodiment, a reaction solution is prepared by bringing a first reagent and a template nucleic acid solution containing a template nucleic acid into contact with each other, and after a nucleic acid is amplified in the reaction solution, a second reagent is added to the reaction solution.

The reagent set according to this embodiment has, for example, the following characteristics.

In the reagent set, a first reagent containing a primer and MgCl₂, and a second reagent containing a probe are included, and the first reagent and the second reagent are separated from each other. Therefore, according to the reagent set, a reaction solution is prepared by bringing the first reagent and a template nucleic acid solution containing a template nucleic acid into contact with each other, and after a nucleic acid is amplified in the reaction solution, the second reagent can be added to the reaction solution. Therefore, according to the reagent set, inhibition of amplification of the nucleic acid by the probe can be suppressed in PCR. Accordingly, by using the reagent set, the thermal cycling speed can be increased (see the below-mentioned “3. Experimental Examples” for the details).

In the case where the probe is added to the reaction solution before a nucleic acid is amplified, when the Tm value of the primer is increased for increasing the PCR speed, it is necessary to set the Tm value of the probe higher than the Tm value of the primer by about 10° C. This is because since hybridization of the probe is performed prior to annealing of the primer, the probe is more reliably degraded by the polymerase. When the Tm value of the probe is increased, the degree of inhibition of amplification of the nucleic acid by the probe is increased. However, the reagent set according to this embodiment is configured such that after a nucleic acid is amplified in the reaction solution, the probe is added to the reaction solution, and therefore, the problem as described above can be avoided, and in principle, as the Tm value or the concentration of the probe is increased, the detection sensitivity can be improved.

Further, according to the reagent set, after a nucleic acid is amplified in the reaction solution, a probe is added to the reaction solution, and therefore, the concentration or the Tm value of the primer can be increased without considering the hybridization of the probe. As the concentration of the primer is higher, the thermal cycling speed can be increased.

In the reagent set, the Tm value of the primer is 70° C. or higher and 80° C. or lower. Therefore, according to the reagent set, the thermal cycling speed can be increased (see the below-mentioned “3. Experimental Examples” for the details).

In the reagent set, the first reagent becomes a reaction solution for performing a nucleic acid amplification reaction, and when the second reagent is added to the reaction solution, the concentration of the divalent cation contained in the reaction solution is 2 mM or more and 7.5 mM or less. Due to this, according to the reagent set, while accelerating the elongation reaction by the polymerase and increasing the PCR speed, nonspecific amplification is suppressed, and a decrease in yield of a specific amplification product can be suppressed.

In the reagent set, the first reagent becomes a reaction solution for performing a nucleic acid amplification reaction, and when the second reagent is added to the reaction solution, the concentration of MgCl₂ contained in the reaction solution may be 4 mM or more and 7.5 mM or less. Due to this, according to the reagent set, while accelerating the elongation reaction by the polymerase and increasing the PCR speed, nonspecific amplification is suppressed, and a decrease in yield of a specific amplification product can be suppressed.

In the reagent set, the first reagent becomes a reaction solution for performing a nucleic acid amplification reaction, and when the second reagent is added to the reaction solution, the concentration of MgSO₄ contained in the reaction solution may be 2 mM or more and 3 mM or less. Due to this, according to the reagent set, while accelerating the elongation reaction by the polymerase and increasing the PCR speed, nonspecific amplification is suppressed, and a decrease in yield of a specific amplification product can be suppressed.

In the reagent set, the probe may contain an MGB molecule. According to this, the reagent set can increase the fluorescence intensity from the probe, and therefore, the amplification amount can be quantitatively determined with high sensitivity (see the below-mentioned “3. Experimental Examples” for the details).

In the reagent set, the probe may be a hydrolysis probe. Due to this, according to the reagent set, in PCR, when the probe is degraded by the polymerase, the quenching effect is cancelled, and the reporter dye emits light, whereby the amplification amount of a nucleic acid can be quantitatively determined.

In the reagent set, the probe may be a non-hydrolysis probe. According to this, in the reagent set, KOD polymerase whose elongation reaction rate is larger than that of Taq polymerase can be used. Therefore, according to the reagent set, the thermal cycling speed can be increased.

In the case where the probe is a non-hydrolysis probe, it is not necessary to degrade the probe in the elongation reaction, and therefore, there is no need to provide an amplification region to which the probe anneals between the forward primer and the reverse primer. According to this, it becomes possible to design the amplification region narrower than in a hydrolysis-type system. Since the amplification region becomes narrower, the annealing time can be reduced, and thus, the thermal cycling speed can be increased.

In the reagent set, with respect to the amount of the primer which anneals to a strand to which a probe hybridizes in the nucleic acid, the ratio of the amount of the primer which anneals to a strand complementary to the strand in the nucleic acid may be more than 1 and less than 4. Due to this, according to the reagent set, when the second reagent containing the probe is added to the reaction solution, the fluorescence intensity from the probe can be increased, and therefore, the amplification amount can be quantitatively determined with high sensitivity (see the below-mentioned “3. Experimental Examples” for the details).

### 2. Nucleic Acid Amplification Reaction Method

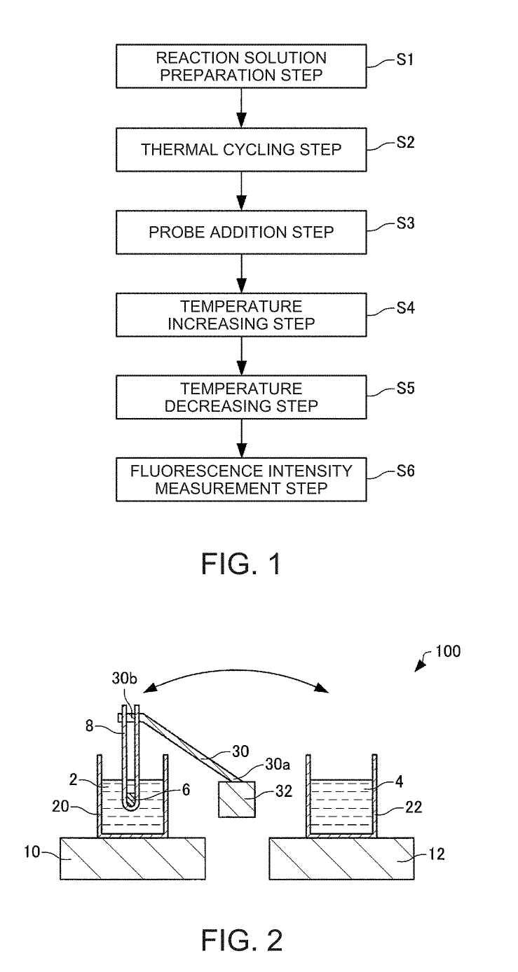

Next, the nucleic acid amplification reaction method according to this embodiment will be described with reference to the accompanying drawings. FIG. 1 is a flowchart for illustrating the nucleic acid amplification reaction method according to this embodiment. Hereinafter, a nucleic acid amplification reaction method using the reagent set according to this embodiment will be described.

First, a reaction solution is prepared by bringing the first reagent of the reagent set according to this embodiment and a template nucleic acid solution into contact with each other (reaction solution preparation step (Step S1)). Specifically, the first reagent is taken out from a case body in which the first reagent and the second reagent are housed, and introduced into a container. Then, a template nucleic acid solution is introduced using a pipette or the like into the container into which the first reagent is introduced so as to bring the first reagent and the template nucleic acid solution into contact with each other, whereby a reaction solution is prepared. The reaction solution contains a component contained in the first reagent.

The template nucleic acid solution is obtained, for example, as follows. That is, a specimen, for example, a cell derived from an organism such as a human or a bacterium, a virus, or the like is collected using a collecting tool such as a cotton swab, and a template nucleic acid is extracted from the specimen using a known extraction method. Thereafter, a template nucleic acid solution is purified so as to have a predetermined concentration using a known purification method. The solution in the template nucleic acid solution is, for example, water (distilled water or sterile water) or a Tris-EDTA (ethylenediaminetetraacetic acid) (TE) solution.

Subsequently, thermal cycling for amplifying a nucleic acid is performed for the reaction solution (thermal cycling step (Step S2)). Here, FIG. 2 is a cross-sectional view schematically showing a thermal cycler 100 for performing thermal cycling for a reaction solution 6 according to this embodiment.

As shown in FIG. 2, the thermal cycler 100 includes a first hot plate 10, a second hot plate 12, a first beaker 20, a second beaker 22, an arm 30, and a fixing section 32.

The first hot plate 10 heats a liquid 2 contained in the first beaker 20 to a first temperature. The first temperature is a temperature suitable for the dissociation (denaturation reaction) of a double-stranded DNA, and is, for example, 85° C. or higher and 105° C. or lower. The type of the liquid 2 is not particularly limited as long as it can be heated to the first temperature by the first hot plate 10, and for example, an aqueous sodium chloride solution and an oil can be exemplified.

The second hot plate 12 heats a liquid 4 contained in the second beaker 22 to a second temperature. The second temperature is lower than the first temperature. The second temperature is a temperature suitable for an annealing reaction and an elongation reaction, and is, for example, 55° C. or higher and 75° C. or lower. That is, in this step, in the heating for the annealing reaction of the primer, the elongation reaction is performed. That is, the annealing reaction and the elongation reaction are performed at the same temperature. The type of the liquid 4 is not particularly limited as long as it can be heated to the second temperature by the second hot plate 12, and for example, an aqueous sodium chloride solution and an oil can be exemplified.

The arm 30 is configured such that one end 30a is fixed by a fixing section 32 and the other end 30b is a free end. The end 30b of the arm 30 supports the container 8 containing the reaction solution 6. The arm 30 is operated by a motor (not shown) such that the end 30b reciprocates arcuately while fixing the end 30a.

By the reciprocation of the arm 30, the reaction solution 6 is alternately placed in the liquid 2 heated to the first temperature and in the liquid 4 heated to the second temperature. According to this, thermal cycling for PCR can be performed for the reaction solution 6. The number of cycles of the thermal cycling in this step can be appropriately set by driving and stopping of the motor, and for example, 20 or more and 60 or less. The conveying time of the reaction solution 6 from the liquid 2 to the liquid 4 and the conveying time of the reaction solution 6 from the liquid 4 to the liquid 2 are, for example, about 0.5 seconds.

In the thermal cycling step (Step S2), a heating time for the denaturation reaction per cycle (in the example shown in the drawing, a time in which the reaction solution 6 is placed in the liquid 2) is, for example, 0.3 seconds or more and less than 2 seconds, preferably 0.5 seconds or more and 0.7 seconds or less. By setting the heating time for the denaturation reaction to 0.3 seconds or more, it is possible to suppress insufficient denaturation due to a too short time for the denaturation reaction. By setting the heating time for the denaturation reaction to less than 2 seconds, the thermal cycling speed can be increased.

In the thermal cycling step (Step S2), a heating time for the annealing reaction and the elongation reaction per cycle (in the example shown in the drawing, a time in which the reaction solution 6 is placed in the liquid 4) is, for example, 6 seconds or less, and 0.3 seconds or more and less than 1 second, preferably 0.5 seconds or more and 0.7 seconds or less. By setting the heating time for the annealing reaction and the elongation reaction to 0.3 seconds or more, it is possible to suppress insufficient annealing reaction and elongation reaction due to a too short time for the annealing reaction and the elongation reaction. By setting the heating time for the annealing reaction and the elongation reaction to 6 seconds or less, the thermal cycling speed can be increased.

In the thermal cycling step (Step S2), a time per cycle of the thermal cycling is 9 seconds or less, preferably 2.5 seconds or less, more preferably 1 second or more and 2.5 seconds or less, further more preferably 2 seconds or more and 2.4 seconds or less. By setting the time per cycle to 9 seconds or less, the thermal cycling speed can be increased. The time per cycle of the thermal cycling includes a time required for the denaturation reaction, the annealing reaction, and the elongation reaction, and the conveying time of the reaction solution for performing these reactions (for example, the conveying time of the reaction solution 6 from the liquid 2 to the liquid 4 and the conveying time of the reaction solution 6 from the liquid 4 to the liquid 2).

The number of bases of a nucleic acid to be amplified in the thermal cycling step (Step S2) may be 200 or less. According to this, the thermal cycling speed can be increased.

Subsequently, the second reagent containing a probe is added to the reaction solution (probe addition step (Step S3)). For example, the reaction solution after thermal cycling is performed is transferred to another container, and then, the probe may be added to the reaction solution by introducing the probe into the container. Alternatively, the probe may be added to the reaction solution by introducing the probe into the container 8 containing the reaction solution.

Subsequently, the temperature of the reaction solution is increased to a temperature (third temperature) at which the amplified nucleic acid is denatured (temperature increasing step (Step S4)). The third temperature in this step is, for example, 85° C. or higher and 105° C. or lower, and may be the same as or different from the first temperature in the thermal cycling step (Step S2).

Subsequently, the temperature of the reaction solution is decreased to a temperature (fourth temperature) at which the probe hybridizes to a single-stranded DNA (denatured nucleic acid) (temperature decreasing step (Step S5)). The fourth temperature in this step may be the same as or different from the second temperature in the thermal cycling step (Step S2).

In the temperature decreasing step (Step S5), the speed of decreasing the temperature of the reaction solution is, for example, 2.5° C./s or more, preferably 12° C./s or more and 200° C./s or less. By setting the speed of decreasing the temperature of the reaction solution to 2.5° C./s or more, the emission intensity from the probe can be increased, and therefore, the amplification amount can be quantitatively determined with high sensitivity (see the below-mentioned “3. Experimental Examples” for the details). By setting the speed of decreasing the temperature of the reaction solution to 200° C./s or less, the cost for the thermal cycler can be kept low.

The temperature increasing step (Step S4) and the temperature decreasing step (Step S5) may be performed in the thermal cycler 100 shown in FIG. 2, or may be performed in a different thermal cycler from the thermal cycler 100. As the different thermal cycler, a device in which a container including a flow channel through which the reaction solution moves can be mounted, and a first region of the flow channel is heated to the third temperature, a second region of the flow channel is heated to the fourth temperature, and the reaction solution can be moved from the first region to the second region by turning the container upside down (by changing a state where the first region is located on the lower side of the second region in the gravitational direction to a state where the second region is located on the lower side of the first region in the gravitational direction) can be exemplified.

Subsequently, the fluorescence intensity of the reaction solution is measured (fluorescence intensity measurement step (Step S6)). A device that measures the fluorescence intensity of the reaction solution is not particularly limited.

The nucleic acid amplification reaction method according to this embodiment has, for example, the following characteristics.

In the nucleic acid amplification reaction method, a thermal cycling step (Step S2), a probe addition step (Step S3), a temperature increasing step (Step S4), and a temperature decreasing step (Step S5) are included. In the nucleic acid amplification reaction method, after a nucleic acid is amplified in a reaction solution, a probe is added to the reaction solution, and therefore, inhibition of amplification of the nucleic acid by the probe can be suppressed in PCR. Therefore, according to the nucleic acid amplification reaction method, the thermal cycling speed can be increased (see the below-mentioned “3. Experimental Examples” for the details).

In the nucleic acid amplification reaction method, in the temperature decreasing step (Step S5), the speed of decreasing the temperature of the reaction solution may be 2.5° C./s or more. Due to this, according to the nucleic acid amplification reaction method, the fluorescence intensity from the probe can be increased, and therefore, the amplification amount can be quantitatively determined with high sensitivity (see the below-mentioned “3. Experimental Examples” for the details).

In the above description, an example in which the probe addition step (Step S3) is performed after the thermal cycling step (Step S2) and before the temperature increasing step (Step S4) is described, however, the probe addition step (Step S3) may be performed after the temperature increasing step (Step S4) and before the temperature decreasing step (Step S5), or may be performed after the temperature decreasing step (Step S5) and before the fluorescence intensity measurement step (Step S6).

### 3. Experimental Examples

Hereinafter, the invention will be more specifically described by showing experimental examples below. However, the invention is by no means limited to the following experimental examples.

### 3.1. First Experimental Example

### 3.1.1. Preparation of Reaction Solution and Experimental Method

As a template nucleic acid (template DNA), a Mycoplasma species DNA was used. The following reaction solution was prepared by adding this template nucleic acid to a reagent.

### Composition of Reaction Solution

| | | |
|---|---|---|
| Platinum Taq polymerase (5 units/μL) | 0.4 | μL |
| Buffer | 2.0 | μL |
| dNTP (10 mM) | 0.25 | μL |
| Forward primer for detection | 0.32 | μL |
| of Mycoplasma species (100 μM) | | |
| Reverse primer for detection | 0.32 | μL |
| of Mycoplasma species (100 μM) | | |
| Mycoplasma species DNA (100 copies/μL) | 1.0 | μL |
| Distilled water | 4.81 | μL |

The reaction solution as described above was placed in a container (Light Cycler Capillaries (20 μL) manufactured by Roche), and PCR was performed by allowing the container to reciprocate 39 times between a high-temperature region (90° C.) and a low-temperature region (66° C.) (see FIG. 2).

Thereafter, the reaction solution was transferred to a different container (MicroAmp Fast Reaction Tubes, manufactured by Applied Biosystems, Inc.), and a probe was added thereto. The container was treated at a high temperature (95° C.) using a Step one Plus Real-time PCR system manufactured by Applied Biosystems, Inc., and thereafter, a fluorescent brightness was measured at a low temperature (55° C.). As the probe, 0.9 μL of a fluorescently labeled probe for detection of a Mycoplasma species (TaqMan (registered trademark) probe manufactured by Sigma-Aldrich Co. LLC.) (10 μM) was used.

The buffer (buffering agent) contains MgCl₂, Tris-HCl (pH 9.0), and KCl. The concentration of MgCl₂ contained in the reaction solution after the probe was added was set to 5 mM. The same applies also to the experimental examples shown below.

The Tm values and the sequences of the primers and the probe used in the first experimental example are as shown in the following Table 1.

TABLE 1

| | Tm | SEQ | |
|---|---|---|---|
| | (° C.) | ID NO: | Sequence |
| Forward | 75.9 | 1 | 5′ GGT GAA ATC CAG GTA |
| primer | | | CGG GTG AAG ACA CC 3′ |
| Reverse | 75.4 | 2 | 5′ GTC CTG ATC AAT ATT |
| primer | | | AAG CTA CAG TAA AGC |
| | | | TTC ACG GGG 3′ |
| Probe | 77.2 | 3 | 5′ FAM-CGG GAC GGA AAG |
| | | | ACC-NFQ-MGB 3′ |

The Tm values of the forward primer and the reverse primer shown in Table 1 were calculated according to the above-mentioned formula (1), and the calculation was performed by setting Ct to 500 nM and Na⁺ to 50 mM in the formula (1).

The Tm value of the probe shown in Table 1 was determined by actual measurement. In the case where the Tm value is determined by actual measurement, a given fluorescent substance is bound to a double-stranded DNA formed by the primer and a complementary strand thereto, and a decrease in the emission intensity from the fluorescent substance due to thermal denaturation is plotted against the temperature. A temperature when a negative primary differential value of this graph reached a peak can be measured as the Tm value.

As Comparative Example, the above-mentioned fluorescently labeled probe was added to the reaction solution in advance, and the reaction solution was placed in a container (Light Cycler Capillaries (20 μL) manufactured by Roche), and PCR was performed by allowing the container to reciprocate 40 times between a high-temperature region (90° C.) and a low-temperature region (66° C.) (see FIG. 2). Thereafter, a fluorescence intensity was measured using a Step one Plus Real-time PCR system.

The time per cycle of the reciprocation (thermal cycling) was set as follows: 2 seconds (high-temperature region: 0.5 sec/low-temperature region: 0.5 sec), 2.4 seconds (high-temperature region: 0.7 sec/low-temperature region: 0.7 sec), and 3 seconds (high-temperature region: 1 sec/low-temperature region: 1 sec).

### 3.1.2. Results of Measurement of Fluorescence Intensity

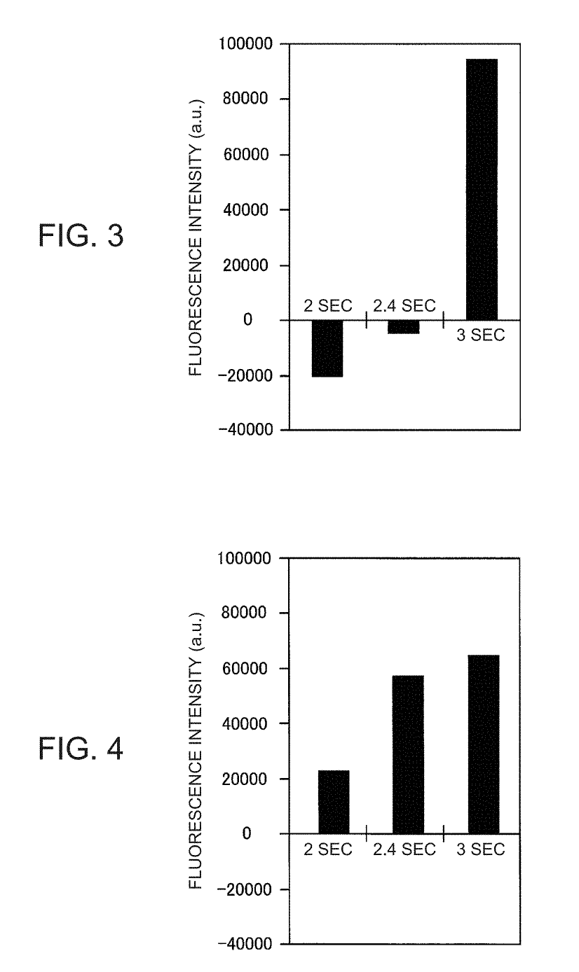

FIG. 3 is a graph showing a fluorescence intensity in the case where the probe was added before amplification of the nucleic acid (also referred to as “pre-addition of probe”) . FIG. 4 is a graph showing a fluorescence intensity in the case where the probe was added after amplification of the nucleic acid (also referred to as “post-addition of probe”).

As shown in FIG. 3, in the case of the “pre-addition of probe”, when the time per cycle of the thermal cycling was 2.4 seconds or less, fluorescence from the probe was not confirmed. On the other hand, as shown in FIG. 4, in the case of the “post-addition of probe”, when the time per cycle of the thermal cycling was 2.4 seconds, fluorescence from the probe was confirmed, and even if the time per cycle was 2 seconds, a large fluorescence intensity of 20000 was confirmed. From FIGS. 3 and 4, it was found that the “post-addition of probe” was very effective in increasing the thermal cycling speed as compared with the “pre-addition of probe”.

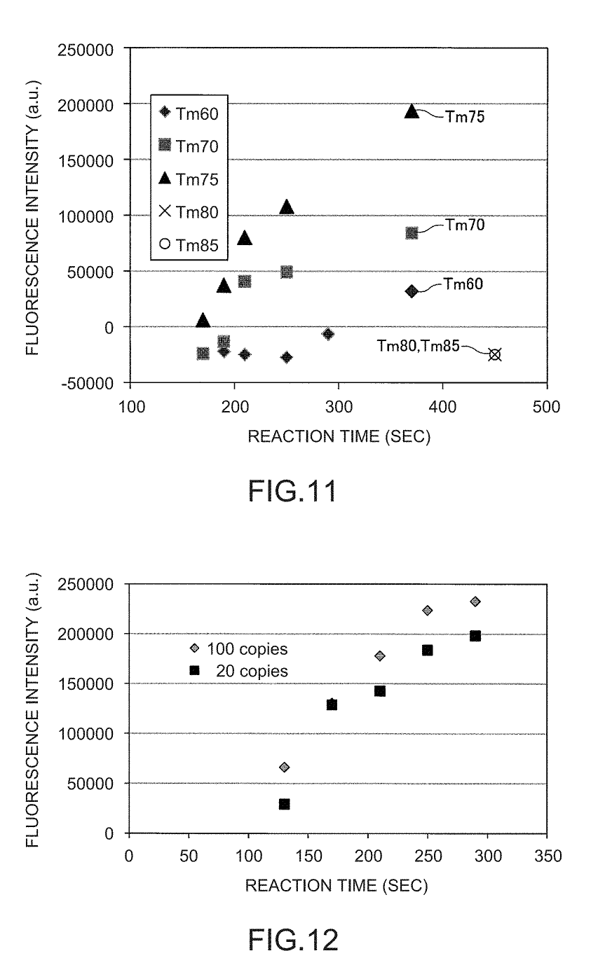

In FIG. 3, a value obtained by subtracting the fluorescent brightness in a state where the nucleic acid was apparently not amplified (for example, after completion of one cycle) from the fluorescent brightness after completion of 40 cycles is plotted. The plot in which the fluorescent brightness shows a negative value is considered to be a measurement error. The same applies also to the below-mentioned FIG. 11.

### 3.1.2. Results of Electrophoresis

The reaction solution of the “pre-addition of probe” described above was analyzed by agarose gel electrophoresis. Further, a reaction solution, to which the probe was not added at all, and for which thermal cycling was performed in the same manner as the reaction solution of the “pre-addition of probe”, was also analyzed by agarose gel electrophoresis.

In addition to the above-mentioned experimental condition, as the probe, a fluorescently-labeled probe (Tm value: 60° C.) which does not contain an MGB molecule among the fluorescently-labeled probes shown in Table 1 was used. Further, also for a case where the time per cycle of the thermal cycling was set to 5 seconds (high-temperature region: 2 sec/low-temperature region: 2 sec), the analysis was performed.

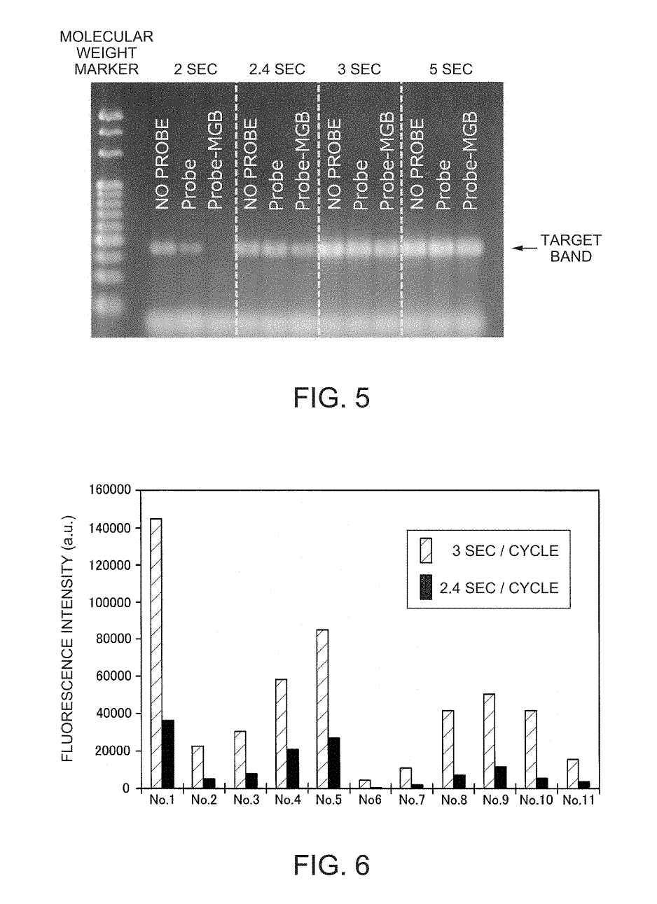

FIG. 5 shows the results of electrophoresis of the pre-addition of probe. In FIG. 5, the “No probe” is a case where the probe was not added at all. The “Probe” is a case where the fluorescently-labeled probe which does not contain an MGB molecule was added. The “Probe-MGB” is a case where the fluorescently-labeled probe which contains an MGB molecule was added.

As shown in FIG. 5, when the time per cycle was 3 seconds or more, a target band (a band of a nucleic acid having a base sequence to be amplified) was clearly confirmed. On the other hand, when the time per cycle was 2.4 seconds, the target band was not clear in the case of “Probe-MGB”, and when the time per cycle was 2 seconds, the target band was not clear or disappeared in the case of “Probe” and “Probe-MGB”. From FIG. 5, it was found that the nucleic acid amplification reaction is inhibited by the probe.

### 3.2. Second Experimental Example

With respect to the “post-addition of probe” used in the first experimental example, the type of the probe was changed, and in the same manner as in the first experimental example, a nucleic acid was amplified, and a fluorescence intensity was measured. The time per cycle of the thermal cycling was set as follows: 2.4 seconds (high-temperature region: 0.7 sec/low-temperature region: 0.7 sec) and 3 seconds (high-temperature region: 1 sec/low-temperature region: 1 sec). The Tm values and the sequences of the probes used in the second experimental example are as shown in the following Table 2.

TABLE 2

| | | Arti- | | |
|---|---|---|---|---|
| | | ficial | SEQ | |
| | Tm | nucleic | ID | |
| | (° C.) | acid | NO: | Sequence |
| No. 1 | 77.2 | — | 3 | 5′ FAM-CGG GAC GGA |
| | | | | AAG ACC-NFQ-MGB 3′ |
| No. 2 | 67 | — | 3 | 5′ FAM-CGG GAC GGA |
| | | | | AAG ACC-BHQ1 3′ |
| No. 3 | 75.25 | LNA | 3 | 5′ FAM-CGG GAC GGA |
| | | | | AAG ACC-BHQ1 3′ |
| No. 4 | 75.25 | LNA | 3 | 5′ FAM-CGG GAC GGA |
| | | | | AAG ACC-BHQ1 3′ |
| No. 5 | 89.45 | LNA | 3 | 5′ FAM-CGG GAC GGA |
| | | | | AAG ACC-BHQ1 3′ |
| No. 6 | 70.26 | LNA | 3 | 5′ FAM-CGG GAC GGA |
| | | | | AAG ACC-BHQ1 3′ |
| No. 7 | 94.92 | LNA | 3 | 5′ FAM-CGG GAC GGA |
| | | | | AAG ACC-BHQ1 3′ |
| No. 8 | 72.25 | ENA | 3 | 5′ FAM-CGG GAC GGA |
| | | | | AAG ACC-BHQ1 3′ |
| No. 9 | 71.26 | ENA | 3 | 5′ FAM-CGG GAC GGA |
| | | | | AAG ACC-BHQ1 3′ |
| No. 10 | 72.76 | BNA | 3 | 5′ FAM-CGG GAC GGA |
| | | | | AAG ACC-BHQ1 3′ |
| No. 11 | 87.2 | PNA | 3 | 5′ FAM-CGG GAC GGA |
| | | | | AAG ACC-BHQ1 3′ |

The Tm values of the probes shown in Table 2 were determined by actual measurement. In the case where the Tm value is determined by actual measurement, a given fluorescent substance is bound to a double-stranded DNA formed by the primer and a complementary strand thereto, and a decrease in the emission intensity from the fluorescent substance due to thermal denaturation is plotted against the temperature. A temperature when a negative primary differential value of this graph reached the peak can be measured as the Tm value.

The No. 3 to No. 11 probes shown in Table 2 each contain an artificial nucleic acid. In the sequences shown in Table 2, each artificial nucleic acid is shown with an underline.

In the second experimental example and the following experimental examples, unless otherwise stated, the reaction solution having the same composition and concentration as those of the reaction solution in the first experimental example was used.

FIG. 6 is a graph showing a fluorescence intensity. In FIG. 6, the shaded bar graph (the bar graph on the left side) of each probe shows a case where the time per cycle of the thermal cycling was set to 3 seconds, and the black bar graph (the bar graph on the right side) of each probe shows a case where the time per cycle of the thermal cycling was set to 2.4 seconds.

As shown in FIG. 6, the probe containing an MGB molecule has the highest fluorescence intensity regardless of the Tm value. Therefore, it was found that the probe containing an MGB molecule is the most suitable probe in the case of the “post-addition of probe”.

As shown in FIG. 6, the No. 6 probe had a low fluorescence intensity. This is considered to be because the 5′ end is an LNA, and therefore, the probability that the probe is degraded by a polymerase is low. Due to this, it can be said that an LNA is preferably located avoiding the 5′ end.

### 3.3. Third Experimental Example

With respect to the “post-addition of probe” used in the first experimental example, the Tm value of the probe was changed to 67° C., 72° C., and 79° C., and in the same manner as in the first experimental example, a nucleic acid was amplified, and a fluorescence intensity was measured. The time per cycle of the thermal cycling was set to 3 seconds (high-temperature region: 1 sec/low-temperature region: 1 sec). As the probe, a hydrolysis probe which does not contain an MGB molecule or an artificial nucleic acid was used.

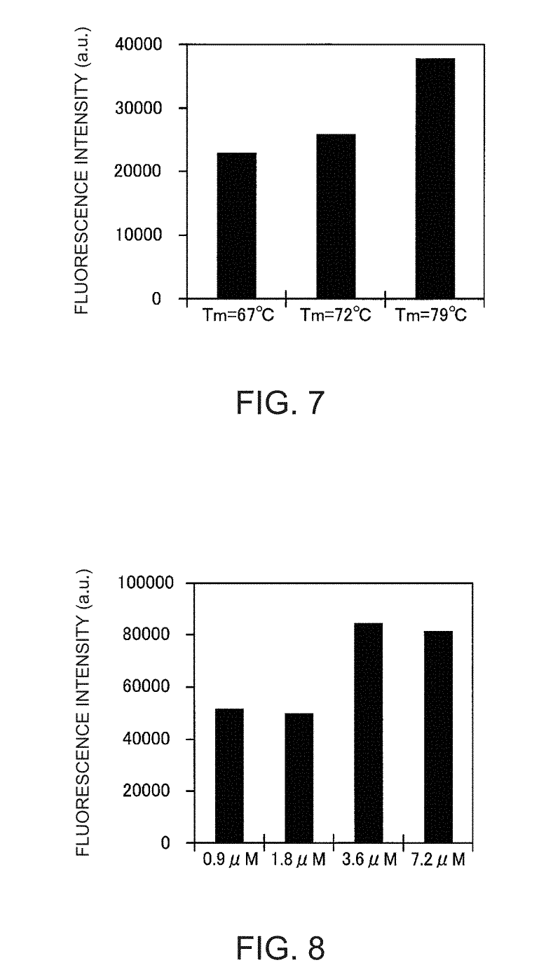

FIG. 7 is a graph showing a fluorescence intensity. From FIG. 7, it was found that in the case of the probe which does not contain an MGB molecule or an artificial nucleic acid, as the Tm value is higher, the fluorescence intensity is higher.

Further, with respect to the “post-addition of probe” used in the first experimental example, the concentration of the probe was changed to 0.9 μM, 1.8 μM, 3.6 μM, and 7.2 μM, and in the same manner as in the first experimental example, a nucleic acid was amplified, and a fluorescence intensity was measured. The time per cycle of the thermal cycling was set to 3 seconds (high-temperature region: 1 sec/low-temperature region: 1 sec). As the probe, a hydrolysis probe which does not contain an MGB molecule or an artificial nucleic acid was used.

FIG. 8 is a graph showing a fluorescence intensity. As shown in FIG. 8, the fluorescence intensity saturated when the concentration of the probe was 7.2 μM. Therefore, it was found that the concentration of the probe was preferably 3.6 μM or less in consideration of the cost.

### 3.4. Fourth Experimental Example

With respect to the “post-addition of probe” used in the first experimental example, the concentration of the primer was changed, and in the same manner as in the first experimental example, a nucleic acid was amplified, and a fluorescence intensity was measured. Specifically, the amount of the forward primer (20 μM) was set to 1.6 μL, and the amount of the reverse primer (100 μM) was set to 0.32 μL, 0.64 μL, 0.96 μL, and 1.28 μL. That is, the ratio (R_{R/F}) of the amount of the reverse primer to the amount of the forward primer was set to 1, 2, 3, and 4. The time per cycle of the thermal cycling was set to 3 seconds (high-temperature region: sec/low-temperature region: 1 sec). As the probe, a hydrolysis probe which contains an MGB molecule was used.

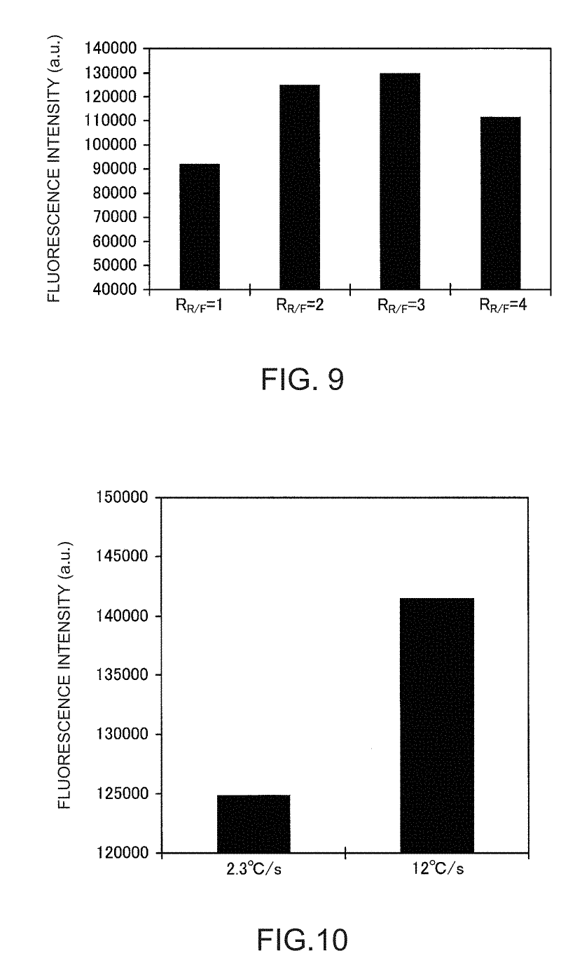

FIG. 9 is a graph showing a fluorescence intensity. As shown in FIG. 9, it was found that by setting the ratio R_{R/F} to more than 1 and less than 4, the fluorescence intensity is increased. It was also found that by setting the ratio R_{R/F} to 2 or more and 3 or less, the fluorescence intensity is more reliably increased. This is considered to be due to the effect of the change in the amounts of the sense and the antisense of the nucleic acid amplification product (amplified nucleic acid or amplicon) by changing the amount of the forward primer and the amount of the reverse primer.

Whether the amount of the forward primer or the amount of the reverse primer is increased varies depending on which of the sense strand or the antisense strand of the nucleic acid sequence the probe binds (hybridizes) to. When the amount of the primer which anneals to a base sequence to which the probe binds is increased to less than 4 times, the fluorescence intensity can be increased.

### 3.5. Fifth Experimental Example

With respect to the “post-addition of probe” used in the first experimental example, in a step of decreasing the temperature of the reaction solution from a high temperature to a low temperature after the probe was added (a step corresponding to the temperature decreasing step (Step S5) in FIG. 1) , the speed of decreasing the temperature of the reaction solution (a temperature decreasing speed) was set to 2.3° C./s and 12° C./s, and a fluorescence intensity was measured.

In the case where the temperature decreasing speed was 2.3° C./s, a step of increasing the temperature of the reaction solution to a high temperature after the probe was added (a step corresponding to the temperature increasing step (Step S4) in FIG. 1) and the step of decreasing the temperature of the reaction solution from a high temperature to a low temperature were performed using the Step one Plus Real-time PCR system in the first experimental example. In the case where the temperature decreasing speed was 12° C./s, after the probe was added, a step of increasing the temperature of the reaction solution to a high temperature and the step of decreasing the temperature of the reaction solution from a high temperature to a low temperature were performed using the device used for amplifying the nucleic acid (see FIG. 2).

The amount of the forward primer (20 μM) was set to 1.6 μL, and the amount of the reverse primer (20 μM) was set to 3.2 μL. Further, the time per cycle of the thermal cycling was set to 3 seconds (high-temperature region: 1 sec/low-temperature region: 1 sec). As the probe, a hydrolysis probe which contains an MGB molecule was used.

FIG. 10 is a graph showing a fluorescence intensity. As shown in FIG. 10, it was found that in the case where the temperature decreasing speed was 12° C./s, the fluorescence intensity was higher than in the case where the temperature decreasing speed was 2.3° C./s. Therefore, it was found that by setting the temperature decreasing speed was 2.5° C./s, the fluorescence intensity can be increased.

The reason why the fluorescence intensity can be increased as the temperature decreasing speed is larger is considered as follows. The Tm value of the nucleic acid amplification product (amplified nucleic acid or amplicon) is higher than the Tm value of the primer or the probe. Due to this, when the temperature decreasing speed is small, the time for annealing only the nucleic acid amplification products to each other becomes long, and annealing of the primer or hybridization of the probe is inhibited. On the other hand, when the temperature decreasing speed is large, the time for annealing only the nucleic acid amplification products to each other becomes short, and therefore, the probability that annealing of the primer or hybridization of the probe is inhibited is decreased. Accordingly, it is considered that as the temperature decreasing speed is larger, the fluorescence intensity is increased.

### 3.6. Sixth Experimental Example

As a template nucleic acid (template DNA), a Mycoplasma species DNA was used. The following reaction solution was prepared by adding this template nucleic acid to a nucleic acid amplification reaction reagent. The sixth experimental example is the case of the “pre-addition of probe”. The nucleic acid amplification reaction reagent is a reagent containing primers, a polymerase, dNTP, a buffer, and a probe.

### Composition of Reaction Solution

| | | |
|---|---|---|
| Platinum Taq polymerase (5 units/μL) | 0.4 | μL |
| Buffer | 2.0 | μL |
| dNTP (10 mM) | 0.25 | μL |
| Forward primer for detection | 1.2 | μL |
| of Mycoplasma species (20 μM) | | |
| Reverse primer for detection | 1.2 | μL |
| of Mycoplasma species (20 μM) | | |
| Fluorescently labeled probe for detection | 0.9 | μL |
| of Mycoplasma species (10 μM) | | |
| Mycoplasma species DNA (100 copies/μL) | 1.0 | μL |
| Distilled water | 3.05 | μL |

As the fluorescently labeled probe, TaqMan (registered trademark) probe manufactured by Sigma-Aldrich Co. LLC. was used.

In the sixth experiment, primers having a different Tm value were used. Specifically, primers having a Tm value of about 60° C. (Tm60), about 70° C. (Tm70), about 75° C. (Tm75), about 80° C. (Tm80), or about 85° C. (Tm85) were used. The Tm values and the sequences of the primers, and the sequence of the probe are as shown in the following Table 3.

TABLE 3

| | | Tm | SEQ | |
|---|---|---|---|---|
| | | (° C.) | ID NO: | Sequence |
| Tm 60 | Forward | 62.6 | 4 | 5′ AAA TCC AGG |
| | primer | | | TAC GGG TGA |
| | | | | AG 3′ |
| | Reverse | 60.6 | 5 | 5′ GTC CTG ATC |
| | primer | | | AAT ATT AAG |
| | | | | CTA CAG TAA |
| | | | | A 3′ |
| Tm 70 | Forward | 70.4 | 6 | 5′ AAA TCC AGG |
| | primer | | | TAC GGG TGA |
| | | | | AGA CAC C 3′ |
| | Reverse | 70.7 | 7 | 5′ GTC CTG ATC |
| | primer | | | AAT ATT AAG |
| | | | | CTA CAG TAA |
| | | | | AGC TTC ACG 3′ |
| Tm 75 | Forward | 75.9 | 1 | 5′ GGT GAA ATC |
| | primer | | | CAG GTA CGG |
| | | | | GTG AAG ACA |
| | | | | CC 3′ |
| | Reverse | 75.4 | 2 | 5′ GTC CTG ATC |
| | primer | | | AAT ATT AAG |
| | | | | CTA CAG TAA |
| | | | | AGC TTC ACG |
| | | | | GGG 3′ |
| Tm 80 | Forward | 80.2 | 8 | 5′ GGT GAA ATC |
| | primer | | | CAG GTA CGG |
| | | | | GTG AAG ACA |
| | | | | CCC G 3′ |
| | Reverse | 79.0 | 9 | 5′ CAT GAT AAT |
| | primer | | | GTC CTG ATC |
| | | | | AAT ATT AAG |
| | | | | CTA CAG TAA |
| | | | | AGC TTC ACG |
| | | | | GGG TC 3′ |
| Tm 85 | Forward | 85.5 | 10 | 5′ GGT GAA ATC |
| | primer | | | CAG GTA CGG |
| | | | | GTG AAG ACA |
| | | | | CCC GTT AGG |
| | | | | CGC 3′ |
| | Reverse | 84.9 | 11 | 5′ GCA TCG ATT |
| | primer | | | GCT CCT ACC |
| | | | | TAT TCT CTA |
| | | | | CAT GAT AAT |
| | | | | GTC CTG ATC |
| | | | | AAT ATT AAG |
| | | | | CTA CAG TAA |
| | | | | AGC TTC ACG |
| | | | | GGG TC 3′ |
| | Probe | | 3 | 5′ FAM-CGG GAC |
| | | | | GGA AAG ACC- |
| | | | | NFQ-MGB 3′ |

The Tm values shown in Table 3 were calculated according to the above-mentioned formula (1), and the calculation was performed by setting Ct to 500 nM and Na⁺ to 50 mM in the formula (1).

10 μL of the reaction solution as described above was placed in a container (Light Cycler Capillaries (20 μL) manufactured by Roche), and PCR was performed by allowing the container to reciprocate between a high-temperature region and a low-temperature region (see FIG. 2). The number of cycles of the thermal cycling was set to 40. Thereafter, the reaction solution was transferred to a different container (MicroAmp Fast Reaction Tubes, manufactured by Applied Biosystems, Inc.), and a fluorescent brightness was measured using a Step one Plus Real-time PCR system manufactured by Applied Biosystems, Inc.

In the PCR using each of the primers (Tm60, Tm70, Tm75, Tm80, and Tm85), the heating temperature (high temperature) for the denaturation reaction was set to 87° C. In the PCR using Tm60, Tm70, Tm75, Tm80, and Tm85, the heating temperature (low temperature) for the annealing reaction and the elongation reaction was set to 60° C., 63° C., 66° C., 69° C., and 72° C., respectively. In the PCR, the heating time (the time at the high temperature) for the denaturation reaction per cycle, the heating time (the time at the low temperature) for the annealing reaction and the elongation reaction per cycle, and the reaction time are shown in the following Table 4. Incidentally, in order to activate the polymerase, the reaction solution was initially heated to the high temperature for 10 seconds (hot start). The reaction time is obtained by, in addition to the polymerase activation time, adding the time at the high temperature and the time at the low temperature multiplied by 40 (the number of cycles), and further adding the conveying time of the reaction solution. Further, in Table 4, the time per cycle of the thermal cycling is obtained by adding the conveying time from the high-temperature region to the low-temperature region (0.5 sec) and the conveying time from the low-temperature region to the high-temperature region (0.5 sec) to the sum of the time at the high temperature and the time at the low temperature. For example, in the case where the reaction time is 370 seconds, the time per cycle of the thermal cycling is as follows: the time at the high temperature (2 sec)+the time at the low temperature (6 sec)+the conveying time from the high-temperature region to the low-temperature region (0.5 sec)+the conveying time from the low-temperature region to the high-temperature region (0.5 sec)=9 sec.

TABLE 4

| | | | | | | | |
|---|---|---|---|---|---|---|---|
| Time at high | 2 | 2 | 2 | 2 | 2 | 2 | 4 |
| temperature (sec) | | | | | | | |
| Time at low | 1 | 1.5 | 2 | 3 | 4 | 6 | 6 |
| temperature (sec) | | | | | | | |
| Reaction time (sec) | 170 | 190 | 210 | 250 | 290 | 370 | 450 |

FIG. 11 is a graph showing a relationship between the reaction time of PCR and a fluorescent brightness. As shown in FIG. 11, in the case where the time at the low temperature was 6 seconds (in the case where the time per cycle was 9 seconds), amplification was confirmed when using Tm60, Tm70, and Tm75, however, in the case where the time at the low temperature was 4 seconds (in the case where the time per cycle was 7 seconds) or less, amplification of a nucleic acid was not confirmed when using Tm60, and amplification was confirmed when using Tm70 and Tm75. Therefore, it was found that by setting the Tm value of the primer to 65° C. or higher and lower than 80° C., even if the time at the low temperature is 4 seconds or less, a nucleic acid can be amplified. This is considered to be because a primer having a higher Tm value anneals to a template nucleic acid faster, and therefore, Tm70 and Tm75 are more suitable for increasing the thermal cycling speed than Tm60. It is also considered that Tm70 and Tm75 have a higher polymerase activity efficiency than Tm60 and can accelerate the elongation reaction, and therefore, Tm70 and Tm75 are more suitable for increasing the thermal cycling speed than Tm60. When considering a variation in the device used in this experimental example, it can be said that when the fluorescence intensity is 35000 or more, a nucleic acid is reliably amplified. Therefore, in the case where the time per cycle is 9 seconds, it cannot be said that a nucleic acid is reliably amplified when using Tm60, and it can be said that a nucleic acid is reliably amplified when using Tm70 and Tm75.

In the above, the sixth experimental example is the case of the “pre-addition of probe”, however, a tendency that “when the Tm value of a primer is set to 70° C. or higher and lower than 80° C., the primer is suitable for increasing the thermal cycling speed” is considered to apply also to the “post-addition of probe”. Therefore, in the case of the “post-addition of probe”, when the Tm value of a primer is set to 70° C. or higher and lower than 80° C., the thermal cycling speed can be increased.

Further, from FIG. 11, it was found that by setting the Tm value of a primer to 70° C. or higher and 75° C. or lower, even if the time at the low temperature is 4 seconds or less, a nucleic acid can be amplified more reliably. Further, in FIG. 11, when using Tm80 and Tm85, amplification of a nucleic acid was not confirmed. This is considered to be because the Tm value was too high, and therefore, a primer dimer or the like was formed.

### 3.7. Seventh Experimental Example

As a template nucleic acid (template DNA), a Bordetella pertussis DNA was used. The following reaction solution was prepared by adding this template nucleic acid to a nucleic acid amplification reaction reagent. The seventh experimental example is the case of the “pre-addition of probe”.

### Composition of Reaction Solution

| | | |
|---|---|---|
| Platinum Taq polymerase (5 units/μL) | 0.4 | μL |
| Buffer | 2.0 | μL |
| dNTP (10 mM) | 0.25 | μL |
| Forward primer for detection | 0.32 | μL |
| of Bordetella pertussis (100 μM) | | |
| Reverse primer for detection of | 0.32 | μL |
| Bordetella pertussis (100 μM) | | |
| Fluorescently labeled probe for | 0.9 | μL |
| detection of Bordetella pertussis (10 μM) | | |
| Bordetella pertussis DNA (20 copies | 1.0 | μL |
| or 100 copies/μL) | | |
| Distilled water | 4.81 | μL |

As the fluorescently labeled probe, TaqMan (registered trademark) probe manufactured by Sigma-Aldrich Co. LLC. was used.

The Tm values and the sequences of the primers, and the sequence of the probe are as shown in the following Table 5.

TABLE 5

| | Tm | SEQ | |
|---|---|---|---|
| | (° C.) | ID NO: | Sequence |
| Forward | 80.8 | 12 | 5′ ATC AAG CAC CGC |
| primer | | | TTT ACC CGA CCT TAC |
| | | | CGC C 3′ |
| Reverse | 80.3 | 13 | 5′ TTG GGA GTT CTG |
| primer | | | GTA GGT GTG AGC GTA |
| | | | AGC CCA 3′ |
| Probe | | 14 | 5′ FAM-AAT GGC AAG |
| | | | GCC GAA CGC TTC A- |
| | | | NFQ-MGB 3′ |

The Tm values shown in Table 5 were calculated according to the above-mentioned formula (1), and the calculation was performed by setting Ct to 500 nM and Na⁺ to 50 mM in the formula (1).

PCR was performed for 10 μL of the reaction solution as described above by performing hot start for 10 seconds in the same manner as in the sixth experimental example. The high temperature was set to 90° C., and the low temperature was set to 60° C. The heating time (the time at the high temperature) for the denaturation reaction per cycle, the heating time (the time at the low temperature) for the annealing reaction and the elongation reaction per cycle, and the reaction time are shown in the following Table 6.

TABLE 6

| | | | | | |
|---|---|---|---|---|---|
| Time at high temperature (sec) | 1 | 2 | 2 | 2 | 2 |
| Time at low temperature (sec) | 1 | 1 | 2 | 3 | 4 |
| Reaction time (sec) | 130 | 170 | 210 | 250 | 290 |

FIG. 12 is a graph showing a relationship between the reaction time of PCR and a fluorescent brightness. As shown in FIG. 12, even if the Tm value of the primer was about 80° C., amplification of a nucleic acid could be confirmed.

The invention includes substantially the same configurations (for example, configurations having the same functions, methods, and results, or configurations having the same objects and effects) as the configurations described in the embodiments. Further, the invention includes configurations in which a part that is not essential in the configurations described in the embodiments is substituted. Further, the invention includes configurations having the same effects as in the configurations described in the embodiments, or configurations capable of achieving the same objects as in the configurations described in the embodiments. In addition, the invention includes configurations in which known techniques are added to the configurations described in the embodiments.

The entire disclosure of Japanese Patent Application No. 2016-149492, filed Jul. 29, 2016 is expressly incorporated by reference herein.

### Sequence Listing Free Text

SEQ ID NO: 1 is the sequence of a forward primer for Mycoplasma bacteria.

SEQ ID NO: 2 is the sequence of a reverse primer for Mycoplasma bacteria.

SEQ ID NO: 3 is the sequence of a fluorescently labeled probe for Mycoplasma bacteria.

SEQ ID NO: 4 is the sequence of a forward primer for Mycoplasma bacteria.

SEQ ID NO: 5 is the sequence of a reverse primer for Mycoplasma bacteria.

SEQ ID NO: 6 is the sequence of a forward primer for Mycoplasma bacteria.

SEQ ID NO: 7 is the sequence of a reverse primer for Mycoplasma bacteria.

SEQ ID NO: 8 is the sequence of a forward primer for Mycoplasma bacteria.

SEQ ID NO: 9 is the sequence of a reverse primer for Mycoplasma bacteria.

SEQ ID NO: 10 is the sequence of a forward primer for Mycoplasma bacteria.

SEQ ID NO: 11 is the sequence of a reverse primer for Mycoplasma bacteria.

SEQ ID NO: 12 is the sequence of a forward primer for Bordetella pertussis.

What is claimed is:

1. A nucleic acid amplification reaction method, comprising:
a thermal cycling step of performing thermal cycling for amplifying a nucleic acid for a reaction solution containing a primer;
a temperature increasing step of increasing the temperature of the reaction solution to a temperature at which the amplified nucleic acid is denatured after the thermal cycling step;
a temperature decreasing step of decreasing the temperature of the reaction solution to a temperature at which a probe hybridizes after the temperature increasing step; and
a probe addition step of adding the probe to the reaction solution, wherein
in the thermal cycling step,
the time per cycle of the thermal cycling is 9 seconds or less,
the Tm value of the primer is 70° C. or higher and 80° C. or lower,
the probe addition step is performed after the thermal cycling step and before the temperature increasing step, or after the temperature increasing step and before the temperature decreasing step, or after the temperature decreasing step.

2. The nucleic acid amplification reaction method according to claim 1, wherein the time per cycle of the thermal cycling is 2.5 seconds or less.

3. The nucleic acid amplification reaction method according to claim 1, wherein the probe contains a minor groove binder molecule.

4. The nucleic acid amplification reaction method according to claim 1, wherein
the reaction solution contains a divalent cation, and
the concentration of the divalent cation contained in the reaction solution after the probe is added is 2 mM or more and 7.5 mM or less.

5. The nucleic acid amplification reaction method according to claim 4, wherein
the reaction solution contains MgCl₂,
the divalent cation is derived from MgCl₂, and
the concentration of MgCl₂ contained in the reaction solution after the probe is added is 4 mM or more and 7.5 mM or less.

6. The nucleic acid amplification reaction method according to claim 4, wherein
the reaction solution contains MgSO₄,
the divalent cation is derived from MgSO₄, and
the concentration of MgSO₄ contained in the reaction solution after the probe is added is 2 mM or more and 3 mM or less.

7. The nucleic acid amplification reaction method according to claim 1, wherein the probe is a non-hydrolysis probe.

8. The nucleic acid amplification reaction method according to claim 1, wherein with respect to the amount of the primer which anneals to a strand to which the probe hybridizes in the nucleic acid, the ratio of the amount of the primer which anneals to a strand complementary to the strand in the nucleic acid is more than 1 and less than 4.

9. The nucleic acid amplification reaction method according to claim 1, wherein in the temperature decreasing step, the speed of decreasing the temperature of the reaction solution is 2.5° C./s or more.

10. A reagent set, comprising:
a first reagent which is a reagent for amplifying a nucleic acid and contains a primer and MgCl₂; and
a second reagent which is a reagent for quantitatively determining the amplification amount of a nucleic acid and contains a probe, wherein
the first reagent and the second reagent are separated from each other,
the Tm value of the primer is 70° C. or higher and 80° C. or lower, and
the first reagent becomes a reaction solution for performing a nucleic acid amplification reaction, and when the second reagent is added to the reaction solution, the concentration of MgCl₂ contained in the reaction solution is 4 mM or more and 7.5 mM or less.

11. A reagent set, comprising:
a first reagent which is a reagent for amplifying a nucleic acid and contains a primer and MgSO₄; and
a second reagent which is a reagent for quantitatively determining the amplification amount of a nucleic acid and contains a probe, wherein
the first reagent and the second reagent are separated from each other,
the Tm value of the primer is 70° C. or higher and 80° C. or lower, and
the first reagent becomes a reaction solution for performing a nucleic acid amplification reaction, and when the second reagent is added to the reaction solution, the concentration of MgSO₄ contained in the reaction solution is 2 mM or more and 3 mM or less.

12. A method of using the reagent set according to claim 10, comprising:
preparing the reaction solution by bringing the first reagent and a template nucleic acid solution containing a template nucleic acid into contact with each other, and
after a nucleic acid is amplified in the reaction solution, adding the second reagent to the reaction solution.

13. A method of using the reagent set according to claim 11, comprising:
preparing the reaction solution by bringing the first reagent and a template nucleic acid solution containing a template nucleic acid into contact with each other, and
after a nucleic acid is amplified in the reaction solution, adding the second reagent to the reaction solution.


SEQUENCE LISTING (14 sequences)

SEQ ID NO: 1 - DNA, 29 bp, Artificial Sequence
  Mycoplasma bacteria Forward primer
ggtgaaatccaggtacgggtgaagacacc

SEQ ID NO: 2 - DNA, 39 bp, Artificial Sequence
  Mycoplasma bacteria Reverse primer
gtcctgatcaatattaagctacagtaaagcttcacgggg

SEQ ID NO: 3 - DNA, 15 bp, Artificial Sequence
  Mycoplasma bacteria Fluorescent probe
cgggacggaaagacc

SEQ ID NO: 4 - DNA, 20 bp, Artificial Sequence
  Mycoplasma bacteria Forward primer
aaatccaggtacgggtgaag

SEQ ID NO: 5 - DNA, 28 bp, Artificial Sequence
  Mycoplasma bacteria Reverse primer
gtcctgatcaatattaagctacagtaaa

SEQ ID NO: 6 - DNA, 25 bp, Artificial Sequence
  Mycoplasma bacteria Forward primer
aaatccaggtacgggtgaagacacc

SEQ ID NO: 7 - DNA, 36 bp, Artificial Sequence
  Mycoplasma bacteria Reverse primer
gtcctgatcaatattaagctacagtaaagcttcacg

SEQ ID NO: 8 - DNA, 31 bp, Artificial Sequence
  Mycoplasma bacteria Forward primer
ggtgaaatccaggtacgggtgaagacacccg

SEQ ID NO: 9 - DNA, 50 bp, Artificial Sequence
  Mycoplasma bacteria Reverse primer
catgataatgtcctgatcaatattaagctacagtaaagcttcacggggtc

SEQ ID NO: 10 - DNA, 39 bp, Artificial Sequence
  Mycoplasma bacteria Forward primer
ggtgaaatccaggtacgggtgaagacacccgttaggcgc

SEQ ID NO: 11 - DNA, 77 bp, Artificial Sequence
  Mycoplasma bacteria Reverse primer
gcatcgattgctcctacctattctctacatgataatgtcctgatcaatattaagctacagtaaagcttcacggggtc

SEQ ID NO: 12 - DNA, 31 bp, Artificial Sequence
  Bordetella pertussis Forward primer
atcaagcaccgctttacccgaccttaccgcc

SEQ ID NO: 13 - DNA, 33 bp, Artificial Sequence
  Bordetella pertussis Reverse primer
ttgggagttctggtaggtgtgagcgtaagccca

SEQ ID NO: 14 - DNA, 22 bp, Artificial Sequence
  Bordetella pertussis Fluorescent probe
aatggcaaggccgaacgcttca